(19) United States
(12) Patent Application Publication
(10) Pub. No.: US 2004/0156873 A1
(43) Pub. Date: Aug. 12, 2004

(54) Topically Bioavailable Acne and Rosacea Treatment Compositions

(76) Inventor: SHYAM K. GUPTA, SCOTTSDALE, AZ (US)

Correspondence Address: SHYAM K. GUPTA, BIODERM RESEARCH, 5221 E. WINDROSE DRIVE, SCOTTSDALE, AZ, 85254, US

(21) Appl. No.: 10/248,691

(22) Filed: Feb. 10, 2003

(51) Int. Cl. A61K 31/728; A61K 31/7008; A61K 31/60; A61K 31/205

(52) U.S. Cl. 424/401; 514/62; 514/160; 514/389; 514/356; 514/54; 514/554

(57) ABSTRACT

The present invention relates to acne and rosacea compositions by a six-prong synergistic combination treatment strategy that includes (1) control of excess sebum production, (2) control of undesirable bacteria or mites, (3) control of inflammation, (4) enhanced desquamation of follicular infundibulum cells, (5) reduction of irritation from anti-acne or rosacea compositions themselves, and (6) enhancement of the topical bioavailability of anti-acne and rosacea compositions. This is achieved by a synergistic combination of commonly utilized topical anti-acne and rosacea ingredients with a topical bioavailability enhancement composition, which results in enhanced anti-acne and rosacea action from such ingredients. Moreover, additional inclusion of an anti-inflammatory composition, and also a vascular micro-circulation enhancement composition, further results in synergistic superior anti-acne and rosacea benefits from such compositions. The present invention discloses additional surprising synergistic combinations for the control of acne and rosacea that are suitable for a variety of delivery systems and packaging forms.

### BACKGROUND OF INVENTION

Acne is a group of diseases whose initial pathology is the comedo and includes acne vulgaris, neonatal acne, infantile acne, and pomade acne. There are approximately 45 million people who suffer from acne in America alone. The disease is so common in youth at their puberty that it often has been termed physiological. Although acne stops appearing for most people by the age of 25, some people, the majority of them are women, experience the disease well into their adult life. This “adult acne” differs from teenage acne in location and that it tends to be more inflammatory with fewer comedones. As the human concern for facial beauty continues to receive heightened marketing attention, the cure for various forms of acne has received much attention, as evidenced by the number of patents and patent applications that have appeared recently. The patent literature abounds with acne treatments. From January 2001 to January 2003 time period over 900 patent applications were published in U.S. Patent Applications computer database search that related to acne. From 1975 to January 2003, there were over 9000 patents issued by the U.S. Patents Office that had some reference to acne. From these data, it would become obvious that a suitable solution to this problem has eluded past efforts.

The disease of acne is characterized by a great variety of clinical lesions. Although one type of lesion may be predominant (typically the comedo), close observation usually reveals the presence of several types of lesions (comedones, pustules, papules, and/or nodules). The lesions can be either noninflammatory or, more typically, inflammatory. In addition to lesions, patients may have, as the result of lesions, scars of varying size. The fully developed, open comedo (i.e., a plug of dried sebum in a skin pore) is not usually the site of inflammatory changes, unless it is traumatized by the patient. The developing microcomedo and the closed comedo are the major sites for the development of inflammatory lesions. Because the skin is always trying to repair itself, sheaths of cells will grow out from the epidermis (forming appendageal structures) in an attempt to encapsulate the inflammatory reaction. This encapsulation is often incomplete and further rupture of the lesion typically occurs, leading to multichanneled tracts as can be seen in many acne scars. In general, there are four major principles presently governing the therapy of acne: (i) correction of the altered pattern of follicular keratinization; (ii) decrease sebaceous gland activity; (iii) decrease the follicular bacterial population (especially P. acnes) and inhibit the production of extra cellular inflammatory products through the inhibition of these microorganisms; and (iv) produce an anti-inflammatory effect. Acne is a chronic inflammatory disease affecting the sebaceous glands. Acne lesions primarily involve the sebaceous glands located on the face, neck, chest and back. Both closed comedones (blackheads) and open comedones (whiteheads) are caused by hyperkeratinization of the infundibulum of the sebaceous duct. These keratinous plugs block the flow of sebum. These dilated ducts abound with the colonies of Priopionibacterium acnes and other fat splitting organisms. The clinically evident open and closed comedones and the microscopic microcomedo are the signal lesions of acne. The acne process results from a cascade of events. First, at puberty a spike in androgen production heralds an increase in sebum production and begins the hyperkeratinization process causing microcomedones and sebum blockade. With this blockage, the number of resident follicular flora increases dramatically. These bacteria produce inflammatory products, which permeate through thin walls of dilated sebum-filled duct. Once in the perifollicular dermis, they trigger the body's own immune defenses (both acute and granulaomatous) to produce the characteristic inflammatory papules, pustules and nodules characteristic of inflammatory acne. The term “acne” is used herein as a general term to include inflammatory diseases of the pilosebaceous unit. In the medical field, the specific type of acne is usually indicated by a modifying term, although the term acne is frequently used alone to designate common acne or acne vulgaris.

Thus, there are four factors that are believed to be the contributors of acne:(1) Increased sebum production; (2) Comedo formation, in which the follicular infundibulum hypercornifies, hyperkeratinizes, and hypodesquamates; (3) Colonization of the follicule by anaerobic Propionibacterium, mainly P. acnes; and (4) The host's inflammatory response.

These four factors are interrelated to each other. Sebum is comedogenic and causes inflammation by itself. The Propionibacterium has high lipolytic activity and liberates free fatty acids from sebum lipids. The free fatty acids have been shown to cause marked inflammation. The microorganisms also produce other extracellular enzymes such as proteases and hyaluronidases, and chemotactic factors, which may be important in the inflammatory process. It would thus be advantageous to provide relief from all of the above four causes of acne.

Rosacea is a common facial dermatitis that currently affects an estimated 13 million Americans. It is a chronic and progressive cutaneous vascular disorder, primarily involving the malar and nasal areas of the face. Rosacea is characterized by flushing, erythema, papules, pustules, telanglectasia, facial edema, ocular lesions, and, in its most advanced and severe form, hyperplasia of tissue and sebaceous glands leading to rhinophyma. Rhinophyma, a florid overgrowth of the tip of the nose with hypervascularity and modularity, is an unusual progression of rosacea of unknown cause. Ocular lesions are common, including mild conjunctivitis, burning, and grittiness. Blepharitis, the most common ocular manifestation, is a nonulcerative condition of the lidmargins. Rosacea most commonly occurs between the ages of 30 to 60, and may be seen in women experiencing hormonal changes associated with menopause. Women are more frequently affected than men; the most severe cases, however, are seen in men.

Fair complexioned individuals of Northern European descent are most likely to be at risk for rosacea; most appear to be pre-disposed to flushing and blushing.

The cause of rosacea is poorly understood, numerous theories have been offered. Hypotheses have included gastrointestinal, psychological, infectious, climatic, and immunological causes, although scientific evidence has not substantiated any of these as primary. Controlled studies have not demonstrated consistent preponderance of gastrointestinal symptoms in rosacea patients. Similarly, neither a distinct psychological abnormality nor one pharmacological mechanism has been isolated in rosacea patients. Perhaps the most commonly touted of the etiologic theories is based on the presence of Demodex folliculorum mites in patients with rosacea; the organism feeds on sebum, and in some cases treatment of demodex infestation has noted improvement in the rosacea; however, in a review of 79 biopsies in 1969, Demodex folliculorum was noted in only 19% of the specimens. A bacterial cause for the disease has been hypothesized, but no consistent findings of one bacterium have been demonstrated. Climate, specifically exposure to extremes of sun and cold, may have an effect on the course of the disease, but the role of climate in what appears to be a connective tissue disorder is not clear. An autoimmune process has been suggested, and tissue fixed immunoglobulins have been reported in patients with chronic inflammation of rosacea, but no other evidence has been found. Other experimental evidence has suggested this disease may represent a type of hypersensitivity reaction. No single hypothesis appears to adequately explain both the vascular changes and the inflammatory reaction seen in rosacea, leaving the pathogenesis unclear. More recently, certain investigators have suggested a connection between rosacea and H. pylori, bacteria shown to cause certain gastrointestinal ulcers, because symptoms seem to have abated in some ulcer patients also suffering rosacea. Nevertheless, the connection between H. pylori and rosacea has been questioned. H. Herr, J. Korean Med Sci Oct. 15, 2000;(S):551-4; R. Boni, Schweiz Med Wochenschr Sep. 16, 2000; 130 (37): 1305-8).

Kang et al. (U.S. Patent Application 20020183399) have recently concluded that rosacea and acne have many common features in their onset and cure. The topical composition for treating rosacea which comprises a combination of an antimicrobial and at least one of (a) an anti-inflammatory and (b) a non-retinoid inhibitor, are very similar for treating acne, according to Kang, for example. It would thus appear logical to develop broad-spectrum compositions that can treat both acne and rosacea, although such compositions are still unknown, until now.

Most acne treatments are directed at preventing inflammatory lesions, particularly the larger nodulo-cystic lesions that tend to be destructive and lead to permanent scarring. In general, visible comedones are the only minor cosmetic nuisances and do not lead to inflammatory lesions. Most acne treatment is directed to four areas: (1) Keratinous plugs in sebaceous ducts; (2) Large sebaceous glands producing excess sebum; (3) Increased numbers of resident follicular bacteria; and (4) Inflammatory response to chemical mediators passing through the follicular wall.

Topical products used to remove comedones are known as comedolytics, the most effective being tretinoin, marketed as a prescription product (Retin A) and by several generic companies. Tretinoin or all-trans retinoic acid is the naturally occurring metabolite of Vitamin A. Tretinoin increases epidermal cell turnover, thus causing comedolysis and most importantly prevents the formation of new keratinous plugs. Applications of tretinoin are normally once a day at bedtime. Dryness, stinging and redness sometimes accompany the applications. Importantly, improvement is usually not seen for 6-8 weeks. Adapalene 0.1% (Differin) is a topical retinoid like tretinoin. Available by prescription only, the gel is usually applied once nightly. Side effects include frequent scaling, burning, redness and dryness. Improvement is delayed and is not evident for 4-8 weeks. Sodium sulfacetamide 10%/sulfur 5% (Sulfacet-R) is also available by prescription only. It is a lotion with antibacterial and comedolytic action. As with tretinoin, improvement is seen in 4-8 weeks. Salicylic acid 2% is an over the counter product that exhibits mild comedolytic activity.

The only products that have anti-sebum activity are estrogens and 1,3 cis-retinoic acid (isotretinoin) and these must be used systemically to be effective. Isotretinoin (Accutane) is a metabolite of Vitamin A available by prescription only. Isotretinoin is used to treat only severe cystic or conglobate acne. Because of its teratogenic properties, birth defects can occur. Isotretinoin is a powerful drug and can elevate triglycerides, total cholesterol and decrease high-density lipoproteins (HDL). Other side effects include dry skin, dry eyes, itching, headaches, nosebleed, and photosensitivity. It is generally taken for 4-5 months to see improvement. Recently, one brand of oral contraceptive has been approved for the treatment of acne for patients who request birth control.

A number of topical and systemic agents are used to lower the number of bacteria that colonize the follicular duct. These include benzoyl peroxide (BP), BP 5%/erythromycin 3% (Benzamycin). BP has antibacterial activity and drying effects and is available over the counter or by prescription. Moreover, it has been recently reported that benzoyl peroxide seems to induce free radical production that can produce skin changes that qualitatively resemble ultraviolet B damage, e.g., increases in epidermal thickness, and deleterious changes in elastin and glycosaminoglycans content (Ibbotson, S. H., et al., J. Inves. Derm., 1999, 12: 933-938). In addition, Benzoyl peroxide is highly reactive, and is thus difficult to stabilize in practical compositions. BP is applied once or twice daily for 1-2 months. BP can produce erythema and peeling of skin. BP is often tried first for both non-inflammatory and mild inflammatory acne. Other topical antibiotics include clindamycin and erythromycin. These are used as solutions, lotions or gels by prescription only. Usually they are applied once or twice daily and results are seen in 1-2 months. Azelaic acid 20% (Azelex) also has mild antibacterial effects.

Systemic antibiotics include tetracycline and its analogs, which are used in low doses for years or until the end of the acne prone years. Most patients with mild inflammatory acne receive a combination of topical antibiotics and tretinoin or other retinoid. Bacterial resistance does occur so antibiotics may be changed or BP is substituted since resistance does not occur with BP. More severe acne requires systemic antibiotics and topical retinoid. The most severe must receive oral isotretinoin for 4-5 months.

There are no drugs that directly affect the inflammatory acne. The retinoids do have some anti-inflammatory properties, but these are poorly described. Topical steroid and even systemic steroids have been used to abort a severe flare of fulminant acne, but these are limited uses because of the side effects. Benzoyl peroxide gels are sometimes used as first aid on acne lesions. These function as a “drawing poultice”, but data supporting this use is not available.

The treatment for acne centers on opening the pore, killing P. acnes, reducing sebum production and regulating inflammatory responses. Retinoids are the agents to reduce sebum production and open the pore. As a topical agent, Differin (adapalene) or Retin-A (tretinoin) is used for mild and moderate acne. Isotretinoin, an oral drug, is very effective but reserved for the severe and resistant acne because of its teratogenicity, hepatotoxicity, elevating triglyceride level and other side effects.

For topical applications, the Food & Drug Administration (FDA) has approved the following ingredients for marketing topical acne products in the USA (Code of Federal Regulations, 21CFR333.310); (1) Resorcinol (2%, in combination only) (2) Resorcinol monoacetate (3%, in combination only) (3) Salicylic acid 0.5 to 2 percent, and (4) Sulfur 3 to 10 percent.

Salicylic acid has been used to treat acne for some time. Salicylic acid dries the skin, which helps in acne management, but it also causes skin irritation in perilesional skin areas of acne patients, especially patients with sensitive skin, and in some cases the erythema is extreme. Salicylic acid is also pH-sensitive, as in neutralized forms, such as sodium salicylate or triethanolamine salicylate, there is a loss of efficacy due to poor bioavailability. In free acid form, salicylic acid is absorbed rapidly and transported into bloodstream. This is the reason for its irritation-causing problems. It would thus be advantageous if salicylic acid can be provided in a form that is slow to absorb into deeper layers of skin for its maximum topical bioavailability and anti-acne efficacy.

Topical and oral antibiotics (especially tetracycline, erythromycin, and clindamycin) are sometimes prescribed for patients with inflammatory papules and pustules, but, in addition to the undesirability of antibiotic overuse in general, which can lead to enhanced susceptibility to infection, disadvantages to such treatments include phototoxicity and interactions with other medications. Other factors that play a role in exacerbating acne, including oil-based cosmetics and some drugs (e.g., androgenic hormones, high-progestin birth control pills, systemic corticosteroids, and iodide- and bromide-containing agents) are often minimized during acne treatment. Besides the side effects of the antimicrobial agents, development of resistant microorganisms has become an important issue nowadays. The number of patients harboring resistant P. acnes has been shown to be growing. For this reason, it would be advantageous to exclude antibiotics and antibacterial agents from topical preparations for acne.

For efficacious topical treatments, it would thus be advantageous to include the following six-prong provisions to control fundamental elements that can provide control of both acne and rosacea in a single composition:(1) Control of excess sebum production, and (2) Control of undesirable bacteria and mites, and (3) Control of inflammation, and (4) Enhanced desquamation of follicular infundibulum cells, and (5) Reduction of irritation from anti-acne and anti-rosacea compositions themselves, and (6) An enhancement of the topical bioavailability of anti-acne and anti-rosacea compositions.

Since the resistance to bacteria is becoming a problem, it would be advantageous to control bacteria without using an antibacterial agent. Also, salicylic acid is being one of the most favored and inexpensive ingredients to control acne, albeit its irritation causing side effects, it would be advantageous to devise methodologies to increase both topical bioavailability and anti-acne efficacy of salicylic acid with a reduction in its irritation causing side effects.

The patent literature abounds with acne and rosacea treatments. From January 2001 to January 2003 time period over 900 patent applications were listed in U.S. Patent Applications computer database search that related to acne. From 1975 to January 2003, there were over 9000 patents issued by the U.S. Patents Office that had some reference to acne. In the same period, there were over 400 patents that had a reference to rosacea. It may also be appreciated that the study and treatment of rosacea has been a long-time concern of the medical community. For example, about 1,000 medical papers have been published on this subject. From these data, it would become obvious that a suitable solution to acne and rosacea problems has not yet been found.

A discussion of the patents and patent applications most pertinent to the present invention follows. U.S. Patent Application 20030021855 (Perricone) discloses acne prevention by the topical application of compositions containing an alkanolamine such as dimethylaminoethanol, in combination with tyrosine and a sulfur ingredient such as lipoic acid or glutathione. Such alkanolamines have strong amine odor that is objectionable to consumers for application on face. Moreover, several such alkanolamines have a high pH that can cause irritation.

U.S. Patent Application 20030021816 (Kang) discloses an immunosuppressant compound, a second active ingredient selected from the group consisting of comedolytics, antibacterials, anti-inflammatory, retinoids, glucocorticoids, and mixtures thereof, and a dermatologically acceptable carrier for acne treatment. Such immunosuppresants are not readily available for common use.

U.S. Patent Application 20020192298 (Burrell) relates to the use of antimicrobial metals, preferably silver for the treatment of an acne. It is preferred that the use of any antimicrobial agents for acne treatment be minimized or eliminated due to development of resistant bacteria.

U.S. Patent Application 20020172672 (Sieberg) is directed to the use of serine proteases, either alone or in combination with a retinoid compound in a pharmaceutical or cosmetic composition for acne treatment. Such enzyme preparations can cause serious skin allergy in some humans.

U.S. Patent Application 20020155180 (Goodman) discloses treatment of acne that comprises topically applying an effective amount of a saw palmetto berry extract and one or more constituents that enhance penetration of the extract into hair follicle sebaceous glands. This disclosure is specific to one ingredient, hence of limited application.

U.S. Patent Application 20020151527 (Wiegand) discloses a method for reducing the number and severity of acne lesions comprising administering a sensory regimen to down regulate the activity of the hypothalamus-pituitary-adrenal axis, in combination with the administration of a topical anti-acne composition comprising an anti-acne agent selected from salicylic acid, sulfur, lactic acid, glycolic acid, pyruvic acid, urea, resorcinol, N-acetylcysteine, retinoic acid, benzoyl peroxide, octopirox, triclosan, azelaic acid, phenoxyethanol, phenoxypropanol, flavinoids, derivatives thereof, and mixtures thereof. The problems of salicylic acid irritation and low topical bioavailability and the use of antibacterials are still not eliminated by Wiegand.

U.S. Patent Application 20010056071 (Pelicchia) discloses the application of antioxidant resveratrol for acne treatment.

U.S. Pat. No. 6,451,773 (Oester et al.) discloses a combination of chitosan with azelaic acid, benzoyl peroxide, retinoic acid, salicylic acid, or mixtures thereof, for the treatment of acne. Chitosan is used as a film-forming agent for topical application of other active ingredients for better adhesion to skin surface. While topical bioavailability is enhanced, the skin irritation and other problems of salicylic acid and azelaic acid use are not reduced.

U.S. Pat. No. 6,440,994 (Sanders) discloses acne treatment using a mixture of antihistamines and anti-inflammatory agents. This does not provide a multifaceted treatment objective.

U.S. Pat. No. 6,436,417 (Singh) discloses solubilized forms of salicylic acid for acne treatment. Such solubilized forms absorb more quickly, reaching bloodstream at a faster rate. Both the topical anti-acne efficacy may be lower and skin irritation may be higher for such compositions.

U.S. Pat. No. 6,433,024 (Popp et al.) discloses topical anti-acne compositions based on benzoyl peroxide, an alpha hydroxy acid, a moisturizer, an isosorbide and a detergent. These compositions contain several skin irritating ingredients.

U.S. Pat. No. 6,365,623 (Perricone) discloses one preferred embodiment that contains a combination of lipoic acid, an .alpha.-hydroxy acid, and dimethylaminoalcohol. Lipoic acid is also claimed to cure rosacea (U.S. Pat. No. 6,472,432; Perricone).

U.S. Pat. No. 6,262,117 (Sefton) discloses acne treatment based on a combination of benzoyl peroxide and azelaic acid. The poor stability of benzoyl peroxide and the skin irritation of either benzoyl peroxide or azelaic acid are still unsolved in Sefton disclosure.

U.S. Pat. No. 6,168,798 (O'Halloran et al.) discloses an alcoholic solution of salicylic acid and salicylates for acne treatment. The rapid absorption of such clear solutions into skin would reduce the topical bioavailability of the active ingredients in such compositions.

U.S. Pat. No. 5,989,523 (Fitzjarrell et al.) discloses a topical spray comprising niacinamide, Aloe Vera extract and NaPCA in a water carrier base. U.S. Pat. No. 5,910,312 (Fried) discloses an anti-acne composition comprising benzoyl peroxide, salicylic acid, and a vasoconstrictor in an inert carrier. Benzoyl peroxide has been suggested for treating acne vulgaris. (See U.S. Pat. No. 4,387,107.)

For many years, benzoyl peroxide has been proven to be a particularly powerful keratolytic and anti-seborrhic agent, as well as being endowed with antibacterial properties. Topical benzoyl peroxide compositions, including a vehicle to enhance the efficacy thereof, are known (See U.S. Pat. No. 4,411,893). Topical compositions of benzoyl peroxide combination with antibiotics are also known. (See U.S. Pat. Nos. 4,407,794; 4,692,329 and 4,387,107).

The problems of skin irritation from benzoyl peroxide or salicylic acid, and the chemical instability and reactivity of benzoyl peroxide are still not solved, although complex, dual-chamber delivery systems (such as U.S. Pat. No. 6,462,025; Vishnupad and U.S. Pat. No. 6,448,233; LaFevre et al.) have been disclosed. Such delivery systems are usually expensive, not-convenient, and not precise in delivering product quantity.

Rosacea is, while rare among colored races, common among races with a light-colored skin, especially among white races, and many cases occur among them. It is divided according to the symptoms into the first degree (telangiectatic rosacea on the forehead, cheeks, dorsum nasi), the second degree (acne rosacea, coexistence of follicular papules and pustules), and the third degree (rhinophyma, dark red tumor and dilated pore on apex nasi). It starts with facial flush (redness) and eventually involves serious impairment of appearance, developing papules, pustules, rhinophyma and tumor on apex nasi, it is also accompanied by seborrhea or enhancement of feeling of heat on the face due to emotional stress or change of environmental temperature. Thus, these symptoms give a patient mental and physical suffering. For the time being, the real cause of rosacea is unknown (Hifuka Chirya Handbook, pp. 380-381, Nanzando (1987) and Gerd Plewing, Albert M. Kligman, ACNE and ROSACEA, 2nd, Completely Revised and Enlarged Edition, pp. 431-454, Springer-Verlag (1993)). Rosacea is apt to be confused with acne. Rosacea, which can coexist with acne, essentially differs from acne. It is characterized by facial flush due to vascularization and proceeds with acne rosacea and tumor on apex nasi. The etiology of rosacea is not fully known, however, at least four factors or co-factors have been suggested. The first of these is endocrine in that the disease occurs most frequently in women between the ages of thirty and fifty. As such, one definite type of rosacea is believed to have a hormonal basis. A second factor is vasomotor lability, believed to have some connection with menopause, which brings about an impairment of normal or consistent flow of blood to the face and its capillaries. Therein, excessive flow of blood to the face, i.e., the well-known “hot flashes” of menopause, is believed to constitute a factor in the disease and its pathogenesis. More particularly, it has been proven that increased skin temperature, as occurs in facial flushing, increases susceptibility to the condition. Rosacea has also been observed as a side effect or immune response to the use of certain cortisone products, which can bring about a severe form of the condition. Finally, pathology analysis of the expressed contents of inflamed pustule follicle of the nose in acute rosacea has demonstrated the existence of demodices, which is a signature of the ectoparasite demodex folliculorum. Accordingly, in such cases, a specific external pathogenic factor is evident. This factor is not present in other forms of acne, e.g., acne vulgaris.

However, the information available so far does establish that both acne and rosacea are interrelated, and hence a common treatment for both would be highly desirable.

Relative to rosacea treatment compositions, U.S. Pat. Nos. 6,352,724 and 5,654,013 (Taylor et al.) discloses rubbing common salt (Sodium chloride). Sodium chloride is the subject of additional disclosures for the treatment of both acne and rosacea (U.S. Pat. No. 4,443,442 to Skillern; U.S. Pat. No. 3,867,522 to Kligman). However, such treatments only work by a single biochemical mechanism, that of abrasion and debridement of the affected skin. Also, once the debridement is completed, the affected skin will feel pain, since it will be equivalent to “adding salt to injury”.

U.S. Pat. No. 6,174,534 (Richard et al.) provides a composition that contains long chain fatty acids for rosacea treatment. Although such composition may be suitable for rosacea, such fatty acids may actually exacerbate acne due to excess sebum-like activity from such fatty acids.

U.S. Pat. No. 6,136,806 (Hittel) discloses certain synthetic organic molecules for rosacea treatment that are not commonly available, or available by prescription only in certain countries.

U.S. Pat. No. 6,133,310 (Parks) and U.S. Pat. No. 5,952,372 (McDaniel) disclose the application of Invermectin in the treatment of rosacea. This ingredient has also been used frequently for the treatment of acne. Invermectin, however, provides relief by a single biochemical mechanism, not a six-prong approach of the present invention. Moreover, Invermectin is not commonly available.

U.S. Pat. No. 5,972,993 (Ptchelintsev) discloses the application of certain antioxidants for the treatment of rosacea. This treatment is thus based only on a single approach of anti-inflammatory action of such antioxidants.

U.S. Pat. No. 5,667,790 (Sellers) discloses the application of aluminum salts for acne and rosacea treatment. Such aluminum salts only block the exudation of sebum and provide relief probably by astringent action. Their long-term use can actually cause additional inflammatory response.

U.S. Pat. No. 5,885,595 (Corey) discloses esters of retinal for acne and rosacea treatment. U.S. Patent Application 20020013361 (Perricone) claims the use of lipoic acid. Since lipoic acid is an antioxidant, it probably works by anti-inflammatory biochemical mechanism, thus constituting just one-prong treatment.

U.S. Patent Applications 20020172719, 20020054918, and 20020041901 (Murad) disclose pharmaceutical composition and methods for the cleansing of skin to facilitate the prevention, treatment, and management of skin conditions that include rosacea and acne by a composition that includes a hydroxy acid or tannic acid to exfoliate a portion of the skin, stabilized hydrogen peroxide to facilitate cleansing of the skin, and an antimicrobial agent to inhibit or reduce microorganisms on the skin. Since the overuse of antimicrobial agents can cause further problems, as mentioned earlier, Murad inventions are thus of limited application, or even to be possibly avoided for any long-term rosacea and acne treatment regimen.

### SUMMARY OF INVENTION

The present invention relates to acne and rosacea treatment compositions by a six-prong approach that includes control of excess sebum production, control of undesirable bacteria or mites, control of inflammation, enhanced desquamation of follicular infundibulum cells, reduction of irritation from anti-acne or rosacea compositions themselves, and enhancement of the topical bioavailability of anti-acne and rosacea compositions. This is achieved by a synergistic combination of commonly utilized topical anti-acne and rosacea ingredients with topical bioavailability enhancement compositions, which results in enhanced anti-acne and rosacea action from such topical anti-acne and rosacea ingredients. Moreover, additional inclusion of an anti-inflammatory composition and also vascular micro-circulation enhancement composition further results in synergistic superior anti-acne and rosacea benefits from such compositions. The present invention discloses additional surprising synergistic combinations for the control of acne and rosacea that are suitable for a variety of delivery systems and packaging forms.

Therefore, the present invention relates to topically bioavailable acne and rosacea treatment compositions comprising: (i) at least one acne and rosacea beneficial cosmetic or drug composition, and (ii) at least one topical bioavailability enhancing antioxidant composition, and, (iii) at least one composition to improve blood micro-circulation, and (iv) a cosmetically or pharmaceutically acceptable delivery system.

In a further respect, this invention relates to the enhancement of anti-acne and anti-rosacea efficacy of certain already known treatment ingredients, such as salicylic acid or azelaic acid, with a concomitant reduction of their irritation potential.

In a further respect, this invention relates to acne and rosacea compositions that contain a topical bioavailability enhancing antioxidant compositions. Surprisingly, such bioavailability enhancing antioxidant compositions, especially those of polyphenols class, can also reduce excess sebum production, and a corresponding reduction in acne or rosacea causing bacterial or mite populations.

In a further respect, this invention relates to the use of blood micro-circulation improvement compositions in combination with acne and rosacea compositions, which surprisingly also help reduce acne problems due to increased blood flow, improved metabolic oxidation of sebum glycerides, and enhanced transport of irritation causing metabolic byproducts from bacterial and mite action away from topical acne and rosacea zones.

In a further respect, this invention relates to synergistic combinations of anti-acne and rosacea composition, a bioavailability enhancing composition that also reduces sebum production, and a blood micro-circulation enhancement composition, to provide full-spectrum acne and rosacea treatment compositions.

In a further respect, this invention relates to topical anti-acne and rosacea compositions that can be formulated in a variety of delivery systems such as lotions, creams, gels, masks, sprays, cleansers, rinses, wipes, scrubs, pads, and such.

### DETAILED DESCRIPTION

The present invention relates to acne and rosacea treatment compositions by a six-prong approach that includes control of excess sebum production, control of undesirable bacteria, control of inflammation, enhanced desquamation of follicular infundibulum cells, reduction of irritation from anti-acne and rosacea compositions themselves, and enhancement of the topical bioavailability of anti-acne and rosacea compositions. This is achieved by a synergistic combination of commonly utilized topical anti-acne and rosacea ingredients (such as salicylic acid or azelaic acid) with a topical bioavailability enhancement composition (such as niacinamide, niacinamide salicylate, niacinamide aerate, or niacinamide lipoate), which results in enhanced anti-acne and rosacea action from such topical anti-acne ingredients. Moreover, additional inclusion of an anti-inflammatory composition, and also vascular micro-circulation enhancement composition, further results in synergistic superior anti-acne and rosacea benefits from such compositions. The present invention discloses additional surprising synergistic combinations for the control of acne and rosacea that are suitable for a variety of delivery systems and packaging forms.

The present invention is based on the following unprecedented six-prong treatment regimen to control both acne and rosacea in a single composition: (1) Control of excess sebum production, and (2) Control of undesirable bacteria and mites, and (3) Control of inflammation, and (4) Enhanced desquamation of follicular infundibulum cells, and (5) Reduction of irritation from anti-acne and anti-rosacea ingredients themselves, and (6) Enhancement of the topical bioavailability of anti-acne and anti-rosacea compositions.

The above six-prong treatment approach of the present invention relates to topically bioavailable acne and rosacea treatment compositions comprising:(i) at least one acne and rosacea beneficial cosmetic or drug composition, and (ii) at least one topical bioavailability enhancing antioxidant composition, and, (iii) at least one composition to improve blood micro-circulation, and (iv) a cosmetically or pharmaceutically acceptable delivery system.

The acne or rosacea beneficial cosmetic or drug composition is selected from a wide choice of compositions that control acne by a variety of biological mechanisms. It is not important to select them on any basis of specific biological action mechanism. Such ingredients and compositions includes, but not limited to, the FDA approved drug active ingredients such as salicylic acid, and cosmetic ingredients such as azelaic acid, and a number of ingredients referenced in prior art compositions. Additional compositions have been disclosed in U.S. patent application Ser. No. 10/280,519 (Filed Oct. 25, 2002; Gupta) and U.S. patent application Ser. No. 10/290,933 (Filed Nov. 7, 2002; Gupta). The amounts can be from about 0.0001% to about 40% of at least one such acne and rosacea beneficial cosmetic or drug composition, preferably from about 0.01 to 20%, and most preferably from about 0.1% to about 10%.

The topical bioavailability enhancing antioxidant composition functions to provide extended bioavailability of acne or rosacea beneficial cosmetic or drug composition at the upper layers of skin. This aspect of topical bioavailability is very important, as most prior art disclosures have mainly concerned with faster, deeper penetrating compositions, which can actually result in their lower efficacy and enhanced irritation. This action further reduces the irritation potential of such compositions. Moreover, such compositions can thus provide extended synergistic benefits over a longer period of time. The amount of at least one topical bioavailability enhancing antioxidant composition can be from about 0.0001% to about 10%, preferably from about 0.01% to about 5%, and most preferably from about 0.1% to about 1%.

The enhancement of blood micro-circulation provides extra oxygen for faster metabolism of any excess sebum oil in the upper layers of skin. This enhanced blood flow also removes any irritation and inflammation causing sebum oxidation fatty acid by-products from acne and rosacea area. The anti-inflammatory action of antioxidant bioavailability enhancing composition is thus synergistically increased. Additionally, increased blood circulation in the micro-capillaries can also stimulate faster cell turnover, further resulting in enhanced desquamation of follicular infundibulum. The amount of at least one composition to improve blood micro-circulation can be from 0.0001% to 10%, preferably from about 0.01% to 5%, and most preferably from about 0.1% to 1%.

The balance of composition can be a cosmetically or pharmaceutically acceptable delivery system. This can be in any suitable form, such as a lotion, cream, gel, spray, thin liquid, body splash, mask, serum, solid cosmetic stick, lip balm, shampoo, liquid soap, bar soap, bath oil, cologne, hair conditioner, salve, collodion, impregnated patch, impregnated strip, skin surface implant, and any other such cosmetically or pharmaceutically acceptable topical delivery forms. The delivery system can further be traditional water and oil emulsions, suspensions, colloids, microemulsions, clear solutions, suspensions of nanoparticles, emulsions of nanoparticles, or anhydrous compositions. The cosmetically or pharmaceutically acceptable delivery system or carrier base can optionally include additional skin beneficial ingredients selected from skin cleansers, surfactants (cationic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectant, humectants, silicones, skin soothing ingredients, analgesics, skin penetration enhancers, solubilizers, moisturizers, emollients, anesthetics, antibacterial agents, antifungal agents, colorants, perfumes, preservatives, seeds, broken seed nut shells, silica, clays, beads, luffa particles, polyethylene balls, mica, pH adjusters, processing aids, and combinations thereof. The amounts of such ingredients are not limited to any specific numbers, as those versed in this art have learned to utilize only safe, effective, and consumer-preferred amounts of such ingredients and compositions.

As can be appreciated from the above disclosures, the present invention covers all aspects of an unprecedented six-prong treatment methodology for the control of acne and rosacea in a single composition. Since acne sufferers also frequently suffer from rosacea, and rosacea sufferer also frequently suffer from acne, such a combination of a single treatment compositions via the six-prong approach of the present invention is also unprecedented and of significant consumer and commercial importance.

### EXAMPLES

The following examples are presented to illustrate presently preferred practice thereof. As illustrations they are not intended to limit the scope of the invention. All quantities are in weight %.

### Example 1

Facial Mask Composition Ingredient % (1) Chitosan 5.0 (2) Lactic Acid 5.0 (3) Glycerin 18.0 (4) Water 70.6 (5) Yohimbine HCl 0.5 (6) Niacinamide Lipoate 0.5 (7) Glutathione 0.2 (8) Preservatives 0.5 Procedure: Mix 1, 2, and 3 to a paste. Mix 4 to 8 separately to a clear solution. Add this to main batch and mix. A clear gel product is obtained. It is applied on the face and neck and left for 10 to 30 minutes, then rinsed off.

### Example 2

Exfoliating Facial Mask Ingredient % (1) Psyllium husk powder 10.0 (2) PEG-630.0 (3) Resveratrol 0.5 (4) Calcium Sulfate 5.0 (5) Soybean Fibers 5.0 (6) Oat Protein 5.0 (7) Niacinamide Salicylate 2.0 (8) Esculin 0.5 (9) Darutoside 0.5 (10) Water 41.0 (11) Preservatives 0.5 Procedure. Mix 1 to 6 to a paste. Mix separately 7 to 11. Add to main batch and mix to a paste. A thin dough-like product is obtained. It is applied on the face and neck area with fingers. After 10 to 30 minutes, it is rubbed with fingers to remove most of the mask components, then rinsed off.

### Example 3

Facial Mask for Prosthetic Delivery Systems Ingredient % (1) Chitosan Ascorbate 10.0 (2) Niacinamide Azelate 2.0 (3) PEG-620.0 (4) Ruscogenins 0.5 (5) Tetrahydrocurcumin 0.1 (6) Licorice Root Extract 0.5 (7) Water 56.4 (8) Soybean Fibers 5.0 (9) Oat Protein 5.0 (10) Preservatives 0.5 Procedure. Mix 1 to 6 to a paste. Mix separately 7 to 10. Add to main batch and mix to a paste. It is applied to the prosthetic device, which is then placed on the face or other body area. After 10 to 30 minutes, prosthetic device is removed, and the mask residue not yet absorbed into the skin is washed off.

### Example 4

Sloughing Mask. This mask is rubber after application to slough-off dead skin cells and rejuvenate fresh skin cells. It is also an exfoliating or Rub-off mask delivery system.

Ingredient % (1) Paraffin wax 25.0 (2) Cetyl alcohol 1.0 (3) Propyl paraben 0.1 (4) Methyl paraben 0.2 (5) GMS-SE 4.0 (6) Stearic acid 3.0 (7) Emulsifying wax 5.0 (8) Deionized water 48.9 (9) Vitamin K 0.3 (10) Pyridoxine Salicylate 0.5 (11) Corn starch 10.0 (12) Polydimethylsiloxane 2.0 Procedure: All ingredients are mixed and heated at 60 to 70C. The mixture is cooled to room temperature. A thick paste is obtained. It is applied on face or body part with fingers. After 10 to 15 minutes, it is rubbed-off with fingers. It comes off as granular particles that contain impurities, dead skin cells, and body oil that has been removed from the body part.

### Example 5

Foaming Facial Mask Ingredient % (1) Chitosan 5.0 (2) Citric acid 5.0 (3) Glycerin 18.0 (4) Water 58.8 (5) Sodium Cocoyl Isethionate 10.0 (6) Niacinamide Retinoate 2.0 (7) Emblica (Phyllanthus emblica) extract 0.2 (8) Horse Chestnut extract 0.5 (9) Preservatives 0.5 Procedure: Mix 1, 2, and 3 to a paste. Mix 4 to 9 separately to a clear solution. Add this to main batch and mix. A thick, translucent gel is obtained. It is applied on the face and neck and left for 10 to 30 minutes, then rinsed off. During rinsing, much foam is generated that provides cleansing action.

### Example 6

Peel-off Facial Mask Composition Ingredient % (1) Deionized Water 76.2 (2) Polyvinyl Alcohol 11.0 (3) Polyethylene Glycol 4.0 (4) Oleth-201.0 (5) Glycerin1.5 (6) Niacinamide Hyaluronate 0.1 (7) Preservative 0.5 (8) Centella Asiatica Extract 0.5 Vitamin A Palmitate 0.1(10) Vitamin E Acetate 0.1 (11) Niacinamide Ascorbate 3.0 (12) Ethylhexyl glycerin 2.0 Procedure: Water was heated at 70 to 80C. All ingredients were added with mixing. The product was cooled to give a translucent yellow syrupy gel. The product is applied to face and neck areas as a thin film. After 10 to 15 minutes, it is peeled-off with fingers.

### Example 7

A Gel Delivery System Composition Ingredient % (1) Deionized Water 41.5 (2) Phenoxyethanol 0.7 (3) Methyl Paraben 0.2(4) Canadian Willow herb 1.1 (5) Chamomile Extract 1.0 (6) Ascorbyl glucosamine 0.1(7) Micromerol 0.0001(8) Helioxine 0.0001 (9) Chlorelline 0.0001 (10) Melarrest-L 0.0001 (11) Green Tea Extract 0.0001(12) Grape seed Extract 0.0001 (13) Vitamin E Acetate 0.0001 (14) Niacinamide Lipoate 0.0001(15) Dimethiconol 2.0(16) Silicone Wax 2.0 (17) Glycerin 49.9992 (18) Xanthan Gum 1.0 (1 g) Aloe Vera 0.2 (20) Fragrance 0.2 Procedure: The carrier base is first made in the standard manner. All other ingredients are then added to the base at room temperature with mixing. A clear gel-like composition is obtained. It is applied on the face and neck area with fingers as a thin film. It is left on the skin to be fully absorbed. It is not rinsed off.

### Example 8

Peel-off Mask Composition Ingredient % (1) Sodium Alginatel 5.0 (2) Polyvinyl alcohol 10.0 (3) Calcium Hydroxycitrate 2.0 (4) Calcium Sulfate1.0 (5) Glycerin 56.4994 (6) Xanthan Gum 0.5 (7) Ascorbic acid 10.0 (8) Niacinamide 5.0 (9) Extract of Rosemary 0.0001(10) Extract of Marigold 0.0001 (11) Extract of Sage 0.0001 (12) Extract of Ginseng 0.0001(13) Extract of St. Johns-wart 0.0001(14) Extract of Ruscus 0.0001 Procedure. Mix polyvinyl alcohol and glycerin and heat at 60 to 70C to a solution. Add all other ingredients and mix. Cool to room temperature. For product application, mix one part of composition with five parts of water. A gel is formed. It is applied to face and neck areas. After 15 minutes, it is peed-off with fingers.

### Example 9

### Acne and Rosacea Treatment Cream Composition

Ingredient % (1) Deionized Water 69.9 (2) Glycerin 2.0 (3) Aloe Vera 0.3 (4) Acrylates/C10-30 Alkyl Acrylate Crosspolymer 0.2 (5) Carbomer 0.2 (6) Sodium Hydroxide 0.2 (7) Sodium Stearyl Phthalamate 1.0 (8) EDTA 0.2 (9) Dimethicone 5.0 (10) Alkyl Benzoate 5.0 (11) GMS-SE 0.5 (12) Cyclomethicone 5.0 (13) Cetyl Alcohol 2.0 (14) Phenoxyethanol 0.7 (15) Parabens 0.3 (16) Cetyl Dimethicone Copolyol 5.0 (17) Vitamin E Acetate 0.2 (18) Centella Asiatica Extract 0.5 (1 g) Tetrahydrocurcumin 0.2 (20) Escin 0.5 (21) Boswellia Serrata Extract 0.5 (22) Niacinamide 0.5 (23) Vitamin K-10.1 Procedure: Mix all ingredients and heat at 60 to 70C. Homogenize, then cool to room temperature. The pH is adjusted to 7.5. An off-white cream is obtained.

### Example 10

### A Facial Cleanser Composition

Ingredient % 1) Glycerin 43.2 (2) Methyl paraben (preservative) 0.2 (3) Niacinamide Ascorbate 5.0 (4) Niacinamide Salicylate 10.0 (5) Deionized Water 15.0 (6) Polyphenols (Red Vine extract) 0.5 (7) Glycyrrhetinic acid 0.5 (8) Phenoxyethanol (preservative) 0.9 (9) Tauranol 1-78-6 (Sodium Cocoyl Isethionate) (surfactant) 20.0 (10) Tauranol ws conc. (Sodium Methyl Cocoyl Taurate) (surfactant) 5.0 (11) Actiplex 2789 (Extract of various plants) 0.1(12) Fragrance 0.5 Procedure: Mix deionized water, ascorbic acid, and niacin in a tank separately. A clear solution is obtained. All of the other ingredients are then added, and the mixture is heated and stirred at 60 to 70 degrees C. for about five to ten minutes until the mixture is homogenous. The homogeneous mixture is cooled to room temperature. A paste-like product is formed.

### Example 11

A high potency serum. Ingredients %(1) Deionized Water 43.0 (2) Propylene Glycol 34.0 (3) Niacinamide Lipoate 10.0 (4) Niacinamide azelate 10.0 (5) Boswellia serrata extract 2.0 (6) Rutin 0.5 (7) Preservative 0.5 Procedure: All ingredients were mixed and heated at 40 to 50C for 30 minutes. The product was cooled. The serum was obtained as a thin solution.

### Example 12

A High Viscosity Serum Delivery System Composition. This can be applied as a soak or gauze also.

Ingredient % (1) Deionized Water (2) 81.5 Glycerin (3) 5.0 Geogard 221 (Preservative) (4) 0.5 Chitosan Lactate 2.5 (5) Chitosan Salicylate 3.0 (6) Chitosan Azelate 2.5 (7) Chitosan Mandelate 1.5 (8) Ginger Root Extract 2.0 (9) Melilot Extract 1.5 Procedure: All ingredients were mixed and heated at 40 to 50C for 30 minutes. The mixture was cooled to room temperature. A clear thin gel was obtained. This can be soaked on fabric or gauze for topical application on acne and rosacea affected zones.

### Example 13

### Facial Powder Delivery System Composition

Ingredient % (1) Corn Starch 96.9 (2) Niacinamide Salicylate 2.0 (3) Pyridoxine Azelate 0.5 (4) Tetrahydrocurcumin 0.1 (5) Carnosine 0.5 Procedure: Mix all ingredients. The powder is applied as a talcum powder on face.

### Example 14

Acne and Rosacea Treatment in A Gel Delivery System. (1) Deionized Water 40.0 (2) Geogard 221 (Preservative) 0.5(3) Dow Corning 2501 Wax 10.00 (4) Structure Plus 4.00 (5) Eyebright Extract 0.1 (6) Botanicals Extracts Blend 0.1 (7) Vitamin E Acetate 0.1 (8) Niacinamide Lactate 2.0 g) Niacinamide Ascorbate 3.0 (10) Niacinamide Salicylate 2.5 (11) Niacinamide Lipoate 1.5 (12) Benzyl nicotinate 1.0 (13) Propylene glycol 35.2 Procedure: All the ingredients were mixed and heated at 60 to 70C for 30 minutes. A yellow gel was obtained.

### Example 15

Acne and Rosacea Facial Cream from a Common Base Composition. Ingredients % (1) Niacinamide 5.0 (2) Carnosine 0.5 (3) Allantoin 0.5 (4) Vitamin K 0.5 (5) Facial cream base 93.5. Mix all ingredients. The cream is applied on the afflicted areas.

I claim:

1. A topical acne and rosacea treatment composition comprising:
(i) at least one acne and rosacea beneficial cosmetic or drug composition, and
(ii) at least one topical bioavailability enhancing antioxidant composition, and
(iii) at least one composition to improve micro-circulation, and
(iv) a cosmetically or pharmaceutically acceptable delivery system.

2. A composition according to claim 1 wherein,
(i) from about 0.0001% to about 40% of at least one acne and rosacea beneficial cosmetic or drug composition, and
(ii) from about 0.0001% to about 10% of at least one topical bioavailability enhancing antioxidant composition, and,
(iii) from about 0.0001% to about 10% of at least one composition to improve micro-circulation, and
(iv) from about 1% to about 99% of a cosmetically or pharmaceutically acceptable delivery system.

3. A composition according to claim 1 wherein topical acne or rosacea beneficial cosmetic or drug composition is selected from Salicylic acid, niacinamide salicylate, niacinamide ascorbate, niacinamide folate, niacinamide lipoate, niacinamide lactate, niacinamide glycolate, niacinamide mandalate, niacinamide malate, niacinamide hydroxycitrate, niacinamide hydroxytetronate, niacinamide aleurate, niacinamide petroselinate, niacinamide pantothenate, niacinamide adenosine monophosphate (AMP), niacinamide diphosphate (ADP), niacinamide adenosine triphosphate (ATP), niacinamide hydroquinone carboxylate, allantoin lactate, allantoin glycolate, allantoin mandelate, allantoin malate, allantoin ascorbate, allantoin phytate, allantoin citrate, allantoin hydroxy citrate, allantoin aleurate, allantoin salicylate, allantoin hyaluronate, glucosamine lactate, glucosamine glycolate, glucosamine malate, glucosamine mandelate, glucosamine ascorbate, glucosamine phytate, glucosamine citrate, glucosamine hydroxy citrate, glucosamine aleurate, glucosamine salicylate, glucosamine hyaluronate, creatine lactate, creatine glycolate, creatine malate, creatine mandelate, creatine ascorbate, creatine phytate, creatine citrate, creatine hydroxy citrate, creatine aleurate, creatine salicylate, creatine hyaluronate, niacinamide lactate, niacinamide glycolate, niacinamide malate, niacinamide mandelate, niacinamide ascorbate, niacinamide phytate, niacinamide citrate, niacinamide hydroxy citrate, niacinamide aleurate, niacinamide salicylate, niacinamide hyaluronate, pyridoxine lactate, pyridoxine glycolate, pyridoxine malate, pyridoxine mandelate, pyridoxine ascorbate, pyridoxine phytate, pyridoxine citrate, pyridoxine hydroxy citrate, pyridoxine aleurate, pyridoxine salicylate, pyridoxine hyaluronate, chitosan lactate, chitosan glycolate, chitosan malate, chitosan mandelate, chitosan ascorbate, chitosan phytate, chitosan citrate, chitosan hydroxy citrate, chitosan aleurate, chitosan salicylate, chitosan hyaluronate, azelaic acid, niacinamide azelate, pyridoxine azelate, chitosan azelate, glucosamine azelate, retinoic acid, niacinamide retinoate, pyridoxine retinoate, chitosan retinoate, glucosamine retinoate, Benzyl Alcohol, Dehydroacetic Acid, Phenoxyethanol, Ethylhexyl glycerin, Usnic acid, or combinations thereof.

4. A composition according to claim 1 wherein the topical bioavailability enhancing antioxidant composition is selected from Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), α-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), pyridoxine, and combinations thereof.

5. A Composition According to claim 1 wherein the blood micro-circulation improvement composition is selected from Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis Vinifera) leaves, apigenin, phytosan, luteolin, and combinations thereof.

6. A composition according to claim 1 wherein cosmetically or pharmaceutically acceptable delivery system or carrier base can optionally include additional skin beneficial ingredients selected from skin cleansers, surfactants (cationic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

7. A composition according to claim 1 wherein a cosmetically acceptable delivery system or a carrier base can be selected in the form of a lotion, cream, gel, spray, thin liquid, body splash, mask, serum, solid cosmetic stick, lip balm, shampoo, liquid soap, bar soap, bath oil, cologne, hair conditioner, salve, collodion, impregnated patch, impregnated strip, skin surface implant, and any other such cosmetically or pharmaceutically acceptable topical delivery forms.

8. The compositions according to claim 7 wherein the cosmetically or pharmaceutically acceptable delivery system can be traditional water and oil emulsions, suspensions, colloids, microemulsions, clear solutions, suspensions of nanoparticles, emulsions of nanoparticles, powders, or anhydrous compositions.
